(19) United States
(12) Patent Application Publication
(10) Pub. No.: US 2018/0318554 A1
(43) Pub. Date: Nov. 8, 2018

(54) MEDICAL ARTICLE SECUREMENT SYSTEM COMPRISING A FLAP HAVING MATING SURFACES OF A MECHANICAL FASTENER ON OPPOSING MAJOR SURFACES

(71) Applicant: 3M INNOVATIVE PROPERTIES COMPANY, St. Paul, MN (US)

(72) Inventors: Naimul Karim, Maplewood, MN (US); Jennifer N. Hanson, Woodbury, MN (US); Shilpi K. Sanghi, Stanford, CA (US); Jia Hu, Mounds View, MN (US)

(21) Appl. No.: 15/755,753

(22) PCT Filed: Oct. 31, 2016
(86) PCT No.: PCT/US2016/059638

§ 371 Date: Feb. 27, 2018

Related U.S. Application Data
(60) Provisional application No. 62/249,525, filed on Nov. 2, 2015.

(51) Int. Cl. A61M 25/02

(52) U.S. Cl. CPC A61M 25/02; A61M 2025/0266; A61M 2025/026

(57) ABSTRACT

A medical article securement system. The system can include a patch comprising a skin-contact adhesive. The system can further include at least one flap comprising a fixed end coupled to the patch, and a free end that is movable with respect to the patch and configured to secure at least a portion of the medical article to the patch. At least the free end of the flap can include a first major surface configured to face the medical article, at least a portion of the first major surface comprising a first mating surface of a mechanical fastener; and a second major surface, opposite the first major surface, configured to face away from the medical article, at least a portion of the second major surface comprising a second mating surface of the mechanical fastener configured to engage the first mating surface of the same flap, a different flap, or both.

### FIELD

The present disclosure generally relates to medical article securement systems comprising a flap having mating surfaces of a mechanical fastener on opposing sides for securing a medical article to the body of a patient, and particularly, for securing medical tubing, such as various catheter systems or other elongated devices to the body of a patient.

### BACKGROUND

Various medical treatments often require the use of medical articles and tubing. In many cases the devices or tubing must be secured to a patient's body. For example, it can be necessary to introduce fluids and liquid medications directly into a blood vessel of a patient. For short term general use, a simple intravenous (IV) catheter can be placed onto a patient's arm. For longer term and more specialized needs, central line catheters or other devices are used. In another example, a urinary catheter (such as a Foley catheter) may be necessary for draining urine from a patient's bladder.

Healthcare providers often secure catheters, other devices or tubing to patients during hospital stays or in-home care. Securing the devices aids in proper positioning, which inhibits dislodgement or tangling that may cause leakage or interruptions in medication dosing. Securement of such devices also minimizes patient discomfort and reduces the risk of infection.

In order to keep a catheter or other medical articles or tubing properly positioned for the duration of treatment, the medical article may be secured to the patient in a variety of ways. One common way of securing a medical article or tubing is by taping the catheter or medical line to the patient's skin. However, taping can be time consuming and labor intensive. Tape can also collect contaminants and must be frequently removed and replaced. In addition, taping is not necessarily effective in securing a medical article or catheter in place, and removal of the tape may cause undesired motion of the device or catheter. Sutures have also been used to attach a catheter to a patient. With sutures, the catheter is stitched onto the skin. Sutures, however, can be a source of infection, can cause pain and inflammation, and can make it more difficult to clean around the incision site. Sutures also require time and skill to place, and can cause scarring.

Various other securement devices have been developed to obviate some of the fallbacks associated with the use of tape and sutures. Some existing securement devices are generally designed for a specific type or size of catheter or medical article. As a result, multiple securement devices may be needed to accommodate different types or sizes of catheters, e.g., in hospitals and clinical settings. This can add to the cost and complexity of sourcing, inventory, storage, and selection of the securement devices. Additionally, many securement devices still suffer effects of patient movement in which tubing may become kinked or pinched and restrict flow of medication, blood, or urine.

There remains a need for securement devices that accommodate varying sizes of medical articles or tubing and allow patient movement while not disrupting or kinking the tubing.

### SUMMARY

The present disclosure is generally directed to medical article securement devices, systems, and methods, and particularly, to universal securement devices, systems, and methods that are adapted to accommodate and reliably secure a large variety of shapes and sizes of catheter systems or other medical articles, particularly elongated medical articles such as medical tubing. The securement devices, systems, and methods of the present disclosure are generally robust, easy to use, and are designed to facilitate coupling and decoupling a medical article to and from the system, while also providing means for reliably retaining a medical article, such as medical tubing (e.g., of a catheter system), for a desired treatment period.

Some aspects of the present disclosure provide a medical article securement system for securing a medical article. The system can include a patch comprising a first major surface, and a second major surface, opposite the first major surface, comprising a skin-contact adhesive. The system can further include at least one flap comprising a fixed end coupled to the patch, and a free end that is movable with respect to the patch and configured to secure at least a portion of the medical article to the patch. At least the free end of the flap can include a first major surface configured to face the medical article, at least a portion of the first major surface comprising a first mating surface of a mechanical fastener; and a second major surface, opposite the first major surface, configured to face away from the medical article, at least a portion of the second major surface comprising a second mating surface of the mechanical fastener configured to engage the first mating surface of the same flap, a different flap, or both.

Other features and aspects of the present disclosure will become apparent by consideration of the detailed description and accompanying drawings.

### BRIEF DESCRIPTION OF THE DRAWINGS

FIG. 1 is a perspective view of a medical article securement system according to one embodiment of the present disclosure, the system shown with a medical article secured.

FIG. 2 is a schematic side cross-sectional view of the medical article securement system of FIG. 1.

FIG. 3A is a schematic side cross-sectional view of a medical article securement system according to another embodiment of the present disclosure, the system shown with a medical article unsecured.

FIG. 3B is a schematic side cross-sectional view of the medical article securement system of FIG. 3A, with the medical article shown secured.

FIG. 4A is a top plan view of a medical article securement system according to another embodiment of the present disclosure, the system shown with a medical article unsecured.

FIG. 4B is a top plan view of the medical article securement system of FIG. 4A, with the medical article shown secured.

FIG. 5A is a top plan view of a medical article securement system according to another embodiment of the present disclosure, the system shown with a medical article unsecured.

FIG. 5B is a top plan view of the medical article securement system of FIG. 5A, with the medical article shown secured.

FIG. 6A is a top plan view of a medical article securement system according to another embodiment of the present disclosure, the system shown with a medical article unsecured.

FIG. 6B is a top plan view of the medical article securement system of FIG. 6A, with the medical article shown secured.

FIG. 7A is a top plan view of a medical article securement system according to another embodiment of the present disclosure, the system shown with a medical article unsecured.

FIG. 7B is a top plan view of the medical article securement system of FIG. 7A, with the medical article shown secured.

### DETAILED DESCRIPTION

The present disclosure generally relates to medical article securement devices and systems and methods for safely and reliably securing a medical article, such as medical tubing, upon a desired location of a patient's body. The medical article securement systems can be universal to accommodate and reliably secure a large variety of medical articles or class of medical articles (e.g., Foley catheters and peripherally inserted central cathers (PICCs)), and can be particularly useful for securing medical articles that need to be secured to a patient over a prolonged period of time, such as weeks or months.

Examples of medical articles that can be employed with the medical article securement devices and systems of the present disclosure include, but are not limited to, medical tubing or fluid supply lines, other similar articles, or combinations thereof. Examples of medical tubing can include, but are not limited to, catheters (e.g., urinary catheters (e.g., Foley catheters), intravenous (IV) catheters, central venous catheters (CVCs), peripherally inserted central catheters (PICCs), arterial catheters, chest tubes, drainage tubes, infant umbilical catheters, and dialysis catheters.

### Definitions

The term “a”, “an”, and “the” are used interchangeably with “at least one” to mean one or more of the elements being described.

The term “and/or” means either or both. For example “A and/or B” means only A, only B, or both A and B.

The terms “including,” “comprising,” or “having,” and variations thereof, are meant to encompass the items listed thereafter and equivalents thereof as well as additional items.

Unless specified or limited otherwise, the term “coupled” and variations thereof are used broadly and encompass both direct and indirect couplings.

Relative positional terms, such as “top,” “bottom,” “upper,” and the like, are only used to describe elements as they relate to one another, but are in no way meant to indicate or imply necessary or required orientations of the apparatus, or to specify how the invention described herein must be used, mounted, displayed, or positioned in use.

The terms “longitudinal” and “axial” are used to refer to a direction or axis that is generally parallel to the direction in which the medical article extends and generally parallel to the overall direction of fluid flow, e.g., along a catheter line.

The term “lateral” is used to refer to a direction or axis that is perpendicular to the longitudinal axis or direction and is used to represent side-to-side motion of a medical article.

The terms “vertical” and “normal” are used to refer to a direction or axis that is normal to both the longitudinal and lateral directions or axes, as well as to the surface of a patient's skin when the medical article securement system is coupled to the patient's skin, and is used to represent the direction of motion toward and away from the skin surface.

The term “proximal” and “distal” are used to represent relative axial directions, relative to a medical practitioner operating or holding the medical article. That is, the term “distal” is used to refer to the direction away from the medical practitioner (and toward an insertion site on the patient and inside the patient's body), and the term “proximal” is used to refer to the direction toward the medical practitioner (and toward the outside of the patient's body, away from the insertion site). For example, the distal end of a catheter is inserted into the patient, while the proximal end extends exterior of the patient toward the medical practitioner. The distal end of the medical article securement system refers to the end of the system that is configured to be oriented toward the distal end of the medical article to which it will be coupled, and the proximal end of the medical article securement system refers to the end of the system that is configured to be oriented toward the proximal end of the medical article.

The terms “layer,” “sheet,” and “dressing,” or variations thereof, are used to describe an article having a thickness that is small relative to its length and width.

The terms “polymer” and “polymeric material” refer to both materials prepared from one monomer such as a homopolymer or to materials prepared from two or more monomers such as a copolymer, terpolymer, or the like. Likewise, the term “polymerize” refers to the process of making a polymeric material that can be a homopolymer, copolymer, terpolymer, or the like. The terms “copolymer” and “copolymeric material” refer to a polymeric material prepared from at least two monomers.

The term “repositionable” refers to the ability of an article or surface to be, at least initially, repeatedly coupled to (e.g., adhered to) and removed from a surface or substrate without substantial loss of coupling capability (e.g., adhesion) and without damage to either surface (e.g., article or underlying substrate) being coupled together. By way of example, some pressure-sensitive adhesives and mechanical fasteners are repositionable.

The phrase “mechanical fastener” or “touch fastener” generally refers to a fastener that includes two mating, or engagement, surfaces configured to be applied to one another, each mating surface having a plurality of engagement structures or features, such that engagement structures on one mating surface are configured to engage with the engagement structures on the opposing mating surface. In some embodiments, the mechanical fastener can include two flexible mating strips or layers. In some embodiments, the mechanical fastener can include a first mating surface comprising tiny, stiff protrusions shaped like hooks that are configured to engage a second mating surface comprising pliable loops (i.e., a “hook and loop fastener,” or “hook and pile fastener”). In some embodiments, the mechanical fastener can include inter-engaging hooks (e.g., self-engaging hooks) on both mating surfaces (i.e., a “hook and hook fastener” or a “self-engaging hook fastener”).

“Peel force” refers to the force needed to “peel” one surface from another surface at an angle with respect to the plane between the surfaces. Adhesive peel force can be measured using the ASTM method referenced in the “Adhesives” section below. Peel force between mating surfaces of a mechanical fastener can be measured using ASTM D5170-98 (2015)—Standard Test Method for Peel Strength (“T” Method) of Hook and Loop Touch Fasteners.

“Shear strength” (or “shear force”) refers to the resistance to forces that cause, or tend to cause, two contiguous parts of a body to slide relatively to each other in a direction parallel to their plane of contact. That is, shear strength is the amount of force required to move one surface relative to another surface when the two surfaces are pulled in opposite directions parallel to their plane of contact. Adhesive shear force can be measured using the ASTM method referenced in the “Adhesives” section below. Shear force between mating surfaces of a mechanical fastener can be measured using ASTM D5169-98(2015)—Standard Test Method for Shear Strength (Dynamic Method) of Hook and Loop Touch Fasteners.

The term “flexible” can generally be used to refer to a material that is drapable. That is, a section of material 5 cm×15 cm when held upright (long end up) folds over under its own weight to drop the opposite end to or below the holder, when performed at ambient conditions. The term “rigid” can generally be used to refer to a material that is essentially non-drapable. That is, a section of material 5 cm×15 cm when held upright (long end up) stands straight up with little or no deflection, when performed at ambient conditions. In some embodiments, rigid materials can show less than 20 degrees of deflection from vertical. “Semi-rigid” materials can be those that exhibit more than 20 degrees of deflection but whose opposite end does not drop below the holder.

The medical article securement systems of the present disclosure include one or more flaps (i.e., flexible flaps), each flap having complementary mating surfaces of a mechanical fastener on opposing sides, such that the flaps can be repositionable as desired, while providing a reliable securement system. As a result, when the flap(s) are wrapped about at least a portion of the medical article (e.g., about at least a portion of a circumference of a medical tubing), one mating surface engages with its complementary mating surface on the opposing side of the same flap, a different flap, or both. As a result, when a secured medical article is subjected to a displacement force (e.g., in a vertical or normal direction), the shear component (i.e., between complementary surfaces of the mechanical fastener, i.e., between adjacent surfaces of the flap) is maximized, while the peel component is minimized. Mechanical fasteners are more resistant to shear forces than peel forces and consequently have higher shear strength than peel strength. As a result, the systems of the present disclosure employ mechanical fasteners on one or more flaps in such a way that the shear strength of the mechanical fastener is exploited.

On the contrary, in some existing systems, a flap may include a mating surface of a mechanical fastener on its underside that can be folded over an article and engaged with a complementary mating surface on a flat surface on which the article resides. In such a configuration, when a secured article is subjected to a displacement force (e.g., in a vertical or normal direction), the peel component (i.e., between complementary surfaces of the mechanical fastener) is maximized, and the overall securement of the article would be reduced, because the shear strength of the mechanical fastener would not be exploited.

In some embodiments, medical article securement systems of the present disclosure can provide a variety of unique advantages and benefits. For example, by employing mechanical fasteners, the systems are able to be opened and closed multiple times (i.e., the securement means are repositionable) while remaining on the patient, which is typically not possible with adhesive tape-based systems. In addition, the fastening means of mechanical fasteners are not compromised if soiled or contaminated (e.g., as compared to adhesive tape-based systems). Furthermore, because systems of the present disclosure do not include rigid hardware structures of fixed positions or orientations, the systems of the present disclosure can accommodate and secure a wide range of sizes and shapes of medical articles. The absence of rigid hardware structures also minimizes patient discomfort and the risk for device-related pressure injuries, such as pressure ulcers.

Furthermore, because the one or more flaps of the systems of the present disclosure include mating surfaces of a mechanical fastener on opposing sides, the flap is configured to be positioned around at least a portion of a medical article and secured in a way that exploits the shear strength of the mechanical fastener, such that the system has a higher resistance to displacement forces on the medical article (e.g., in a vertical or normal direction).

In addition, because the one or more flaps of systems of the present disclosure include mating surfaces of a mechanical fastener on opposing sides, no matter how the flap(s) are positioned around the medical article to secure it, enhanced securement will be provided (i.e., the shear strength of the mechanical fastener will be exploited), no matter how the flap either contacts itself, or how the multiple flaps contact each other, thereby making the systems of the present disclosure more fool-proof, consistent, and user-independent.

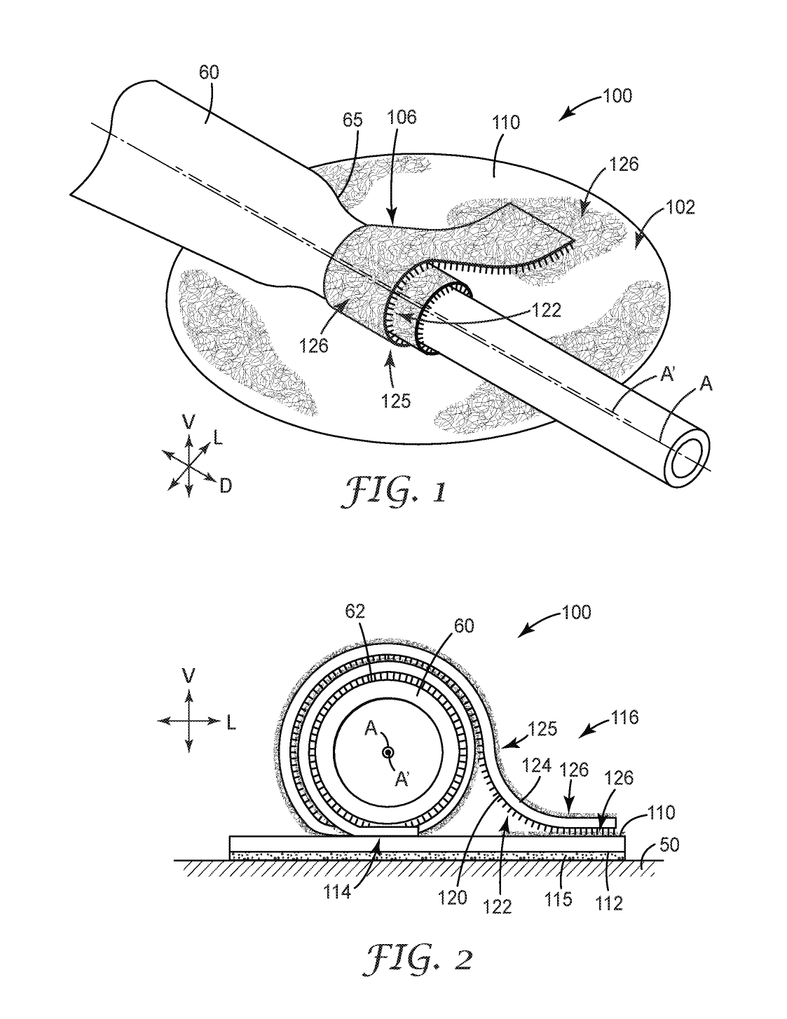

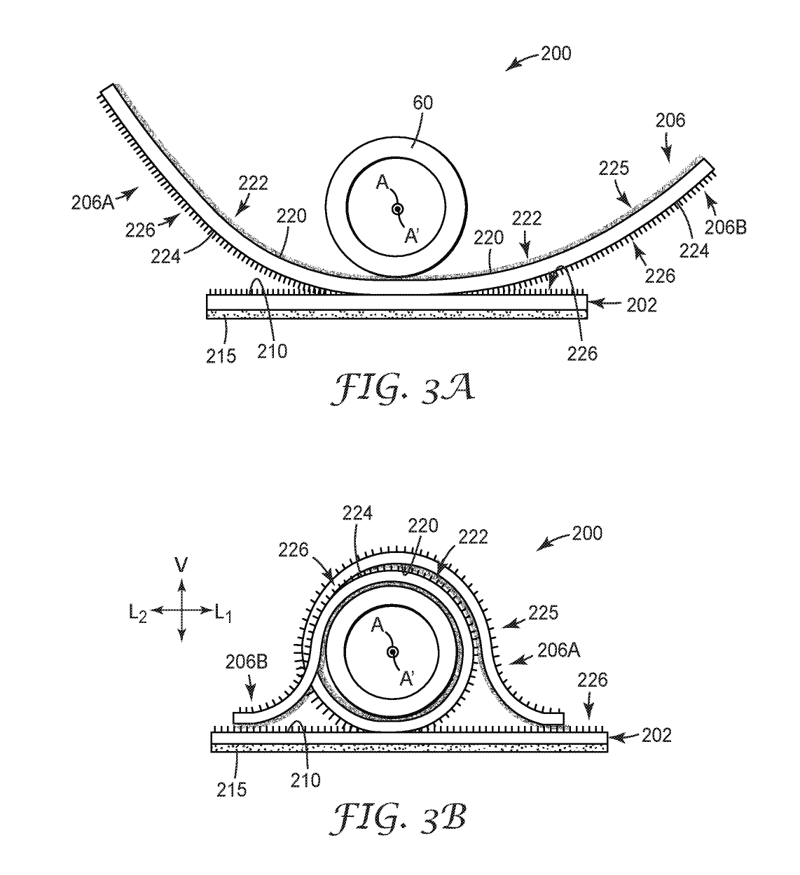

FIGS. 1-2 illustrate a medical article securement system 100 according to one embodiment of the present disclosure. The medical article securement system 100 is shown as securing a medical article 60, which is shown by way of example as being medical tubing, such as a catheter. The medical article 60 has a longitudinal axis A that defines a longitudinal direction D. The system 100 has a longitudinal axis A′ that is configured to be substantially aligned with the longitudinal axis A of the medical article 60 when the medical article 60 is positioned on or coupled to the system 100.

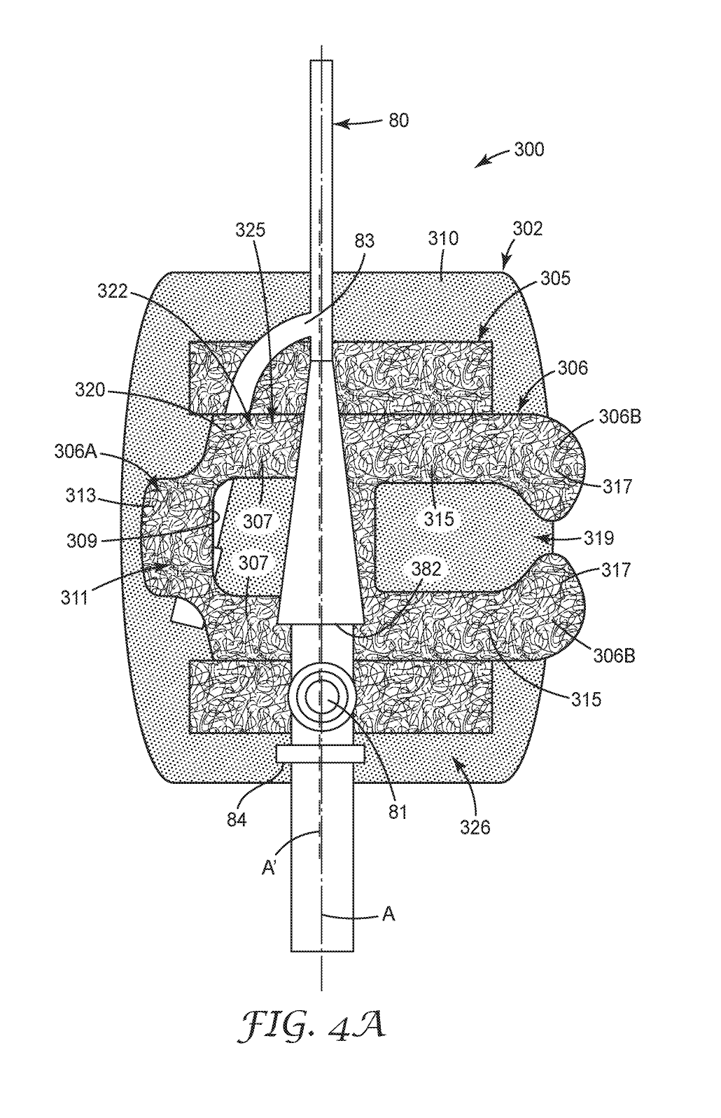

As shown in FIGS. 1-2, the system 100 can include a patch (or base dressing, or base layer) 102 configured to receive the medical article 60 such that the longitudinal axis A of the medical article 60 is substantially aligned with the longitudinal axis A′ of the system 100, and one or more flaps 106. The system 100 of FIGS. 1-2 is shown as including only one flap 106; however, other embodiments of systems of the present disclosure include multiple flaps, as described in greater detail with respect to FIGS. 3A-7B. When describing the system 100 of FIGS. 1 and 2, one flap 106 will be described for simplicity, but it should be understood that the description can apply to as many flaps that are employed in a given system.

The flap 106 can be coupled to the patch 102 and/or integrally formed with the patch 102, and the patch 102 can be adhered to skin. The flap 106 can be used to secure the medical article 60 to the system 100 and the patient's skin. As shown in FIGS. 1 and 2, in some embodiments, the flap 106 can be positioned with respect to the patch 102 and oriented with respect to the longitudinal direction D, such that the flap 106 can secure the medical article 60 in such a way that it inhibits movement of the medical article 60 in at least a vertical direction V, but also potentially in a lateral direction L.

In some embodiments, the flap 106 and the patch 102 can be formed of the same backing material. In some embodiments, the flap 106 may be formed of a different backing material than the patch 102. Various additional details regarding backings of the present disclosure are described in greater detail below under the section entitled, “Backings.”

The patch 102 includes a first major surface 110 configured to face away from the patient's skin, and a second major surface 112 (see FIG. 2) opposite the first major surface 110 that comprises a skin-contact adhesive 115 for adhering to the skin 50 (see FIG. 2). Although the patch 102 is illustrated as being substantially round or oval, it should be understood that the patch 102 can take on a variety of shapes and sizes, depending on the shapes and configurations of the other elements of the system 100 and the medical article 60 to be coupled to the system 100. In some embodiments, the patch 102 comprises a laminated structure comprising one or more of a fabric, a woven fibrous web, a nonwoven fibrous web, a knit, a polymeric film, or combinations thereof.

The skin-contact adhesive 115 is generally a pressure-sensitive adhesive, and particularly is a pressure-sensitive adhesive that is capable of securely but releasably adhering or bonding to skin (e.g., mammalian skin). The skin-contact adhesive 115 is also generally safe and non-toxic. Skin-contact adhesive layers will generally be selected according to the desired end use of the patch 102. In some embodiments, the patch 102 can include more than one skin-contact adhesive 115. Where the patch 102 comprises more than one skin-contact adhesive layer 115, each skin-contact adhesive layer 115 may be selected independently of each other with regard to material and thickness used. Examples of suitable adhesives include acrylates, silicones, polyisobutylenes, synthetic rubber, natural rubber, and copolymers and mixtures thereof. Acrylates and silicones can be preferred skin-contact adhesives 115. In general, the skin-contact adhesive 115 should cause little or no irritation or sensitization of the skin during the intended wear period. Examples of skin-contact adhesives 115 that can be employed with the systems of the present disclosure include, but are not limited to, the adhesives described in U.S. Pat. Nos. RE24,906; 3,389,827; 6,103,369 and 4,499,896, which are incorporated herein by reference. In addition, silicone adhesives such as those described in U.S. Patent Publication No. 2011/0212325, which is incorporated herein by reference, can also be employed.

In some embodiments, e.g., in embodiments employing silicone adhesives, the patch 102 and the skin-contact adhesive 115 can be perforated to provide openings from the first major surface 110 of the patch 102 all the way through the second major surface 112 and the skin-contact adhesive 115, which can enhance permeability of the patch 102 and can minimize moisture build-up at the skin surface underlying the patch 102.

In some embodiments, the system 100 can further include one or more release liners that can provide a release layer or surface to the skin-contact adhesive 115 on the second major surface 112 of the patch 102 prior to use. Examples of liners suitable for use with systems of the present disclosure can include, but are not limited to, kraft papers, polyethylene, polypropylene, polyester, or combinations thereof. Such liners can be coated with release agents, such as fluorochemicals, silicones, or other suitable low surface energy materials. Other adhesives and release liner combinations known to those of ordinary skill in the art can be employed in the systems of the present disclosure.

The flap 106 can be coupled to the patch 102 using a variety of coupling means including, but not limited to, one or more of adhesives, cohesives, welding (e.g., sonic [e.g., ultrasonic] welding), any thermal bonding or heat sealing technique (e.g., heat and/or pressure applied to one or both of the components to be coupled), other suitable coupling means, or combinations thereof. Additionally or alternatively, in some embodiments, at least a portion of the flap 106 can be integrally formed with the patch 102.

The flap 106 of the present disclosure can be flexible so as to easily accommodate and secure the medical article 60, or any medical article of interest, and so as to be able to be wrapped about at least a portion of the medical article 60. For example, in some embodiments, the medical article 60 can include medical tubing, and the flap 106 can be wrapped about at least a portion of a circumference of the medical tubing.

As shown in FIG. 2, the flap 106 includes a fixed end 114 coupled to the patch 102, and a free end 116 that is movable with respect to the patch 102 and configured to be wrapped around (i.e., has a sufficient length to be wrapped around) at least a portion of the medical article 60, and in some embodiments, configured to overlap, or cross, the longitudinal axis A′ of the system 100 (and the longitudinal axis A of the medical article 60) to secure at least a portion of the medical article 60 to the patch 102 (i.e., to the first major surface 110 of the patch 102).

The flap 106 can be movable with respect to the patch 102 (and/or the medical article 60) between (i) a first position in which the flap 106 is open and the medical article 60 is not secured, and particularly, in which at least a portion of the free end 116 is not positioned in an overlapping relationship with the medical article 60, and (ii) a second position (see FIGS. 1 and 2) in which the flap 106 is closed and the medical article 60 is secured, and particularly, in which at least a portion of the free end 116 is positioned in an overlapping relationship with at least a portion of the medical article 60.

The flap 106, and particularly, at least the free end 116 of the flap 106, can include a first major surface 120 (see FIG. 2) configured to face the medical article 60 (i.e., at least when the free end 116 of the flap 106 is in the second position); and a second major surface 124 (see FIG. 2), opposite the first major surface 120, configured to face away from the medical article 60 (i.e., at least when the free end 116 of the flap 106 is in the second position). At least a portion of the first major surface 120 can include a first mating surface 122 of a mechanical fastener 125. At least a portion of the second major surface 124 can include a second mating surface 126 of the mechanical fastener 125 configured to engage the first mating surface 122 of the same flap 106, a different flap, or both, when the flap(s) 106 are coupled to the medical article 60 (i.e., to secure the medical article 60 in a way that exploits the shear strength of the mechanical fastener 125).

By way of example only, the first mating surface 122 is schematically illustrated as including hooks or pegs, and the second mating surface 126 is schematically illustrated as including piles or loops. However, it should be understood that any appropriate mechanical fastener engagement surface or mechanism can be employed, as long as the first mating surface 122 and the second mating surface 126 are complementary mating surfaces of a mechanical fastener that are configured to engage one another.

As shown in FIG. 2, the first mating surface 122 and the second mating surface 126 are positioned to engage one another in a way that exploits the shear strength of the mechanical fastener 125, because each major surface of the flap 106 includes a complementary mating surface of the mechanical fastener.

By way of example, in some embodiments, the first mating surface 122 and the second mating surface 126 are positioned to engage one another over, or in the region of, at least an upper surface 62 of the medical article 60 (i.e., opposite the patch 102), when the flap 106 is coupled to, or wrapped around at least a portion of, the medical article 60. As a result, when a displacement force is exerted on the medical article 60, e.g., in the vertical direction V (see FIG. 2), the shear strength of the mechanical fastener will be exploited, rather than simply subjecting the mechanical fastener to a peel force, resulting in very reliable securement of the medical article 60.

As shown in FIGS. 1 and 2, in some embodiments, the flap 106 (or flaps, if more than one is employed) can extend laterally in a lateral direction L. In some embodiments, the flap 106 can be elongated in the lateral direction L, but this need not be the case. That is, a flap 106 can extend laterally as long as the flap 106 is oriented to include a dimension in the lateral direction L. As a result, the flap 106 can be positioned to wrap laterally about a medical article 60, with respect to the longitudinal axis A of the medical article 60.

For example, in some embodiments, the flap 106 can be movable with respect to the patch 102 (and/or the medical article 60) between (i) a first position in which the flap 106, and particularly, the free end 116 of the flap 106, does not overlap the longitudinal axis A′ of the system 100 (or the longitudinal axis A of the medical article 60); and (ii) a second position in which the flap 106, and particularly, the free end 116 of the flap 106, overlaps the longitudinal axis A′ of the system (or the longitudinal axis A of the medical article 60). Said another way, in some embodiments, the flap 106 can be movable with respect to the patch 102 (and/or the medical article 60) between a first position in which the free end 116 does not cross (e.g., laterally cross) the longitudinal axis A′ of the system 100 (or the longitudinal axis A of the medical article 60) and a second position in which the free end 116 crosses (e.g., laterally cross) the longitudinal axis A′ of the system (or the longitudinal axis A of the medical article 60).

In some embodiments (e.g., embodiments employing only one flap 106 such as that of FIGS. 1 and 2), the flap 106 can be configured (e.g., sized and shaped) to be wrapped about the medical article 60 (e.g., about the circumference of medical tubing) at least one time, such that the flap 106 can be wrapped upon itself over the medical article 60, i.e., such that a first portion of the free end 116 of the flap 106 overlaps another portion of the free end 116 of the flap 106. In such embodiments, the first mating surface 122 can engage the second mating surface 126, providing the exploitation of the shear strength of the mechanical fastener 125.

In other embodiments, e.g., those employing more than one flap 106, each flap 106 may only be configured to overlap the medical article 60 (e.g., the upper surface 62 thereof), but the flaps 106 are shaped, sized and positioned relative to one another to ensure that the first mating surface 122 of one flap 106 overlaps and engages with the second mating surface 126 of one or more other flaps 106, and optionally also the same flap 106, when in the second position, such that the shear strength of the mechanical fastener 125 is exploited. Such embodiments will be described in greater detail below with respect to FIGS. 3A-7B.

In some embodiments, the system 100 can be configured to secure the medical article 60 in the region of an irregular feature to enhance securement of the medical article 60. That is, in some embodiments, the system 100 can be configured (e.g., the flap 106 can be shaped, sized and/or positioned) to facilitate coupling to an irregular feature of the medical article 60, for example, by being coupled to a portion of the medical article 60 that comprises the irregular feature or by being coupled to a portion of the medical article 60 adjacent the irregular feature. For example, as shown in FIG. 1, the medical article 60 includes medical tubing that includes a change in diameter 65. As a result, it can be useful to secure a portion of the medical article 60 comprising the irregular feature, e.g., by coupling the flap 106 to the medical article 60 adjacent the change in diameter 65. Such positioning of the flap 106 and the medical article 60 relative to one another can further secure the medical article 60 by inhibiting movement of the medical article 60 not only in the vertical direction V and the lateral direction L, but also in the longitudinal direction D, or at least in one of a proximal or a distal longitudinal direction.

Medical articles can include other irregular features over or adjacent which it can be useful to position the flap 106. For example, an irregular feature of a medical article can include, but is not limited to, a connection point for multiple lumens (e.g., bifurcation point, trifurcation point, etc.), a change in diameter (e.g., a step-change or multi-step change), a protrusion (e.g., a knob, a dial, a meter, a connector), a constriction, or any other feature where a medical article may deviate from a uniform or regular shape, such as a tube or cylinder having a substantially uniform diameter.

As further shown in FIGS. 1 and 2, in some embodiments, the patch 102, and particularly, the first major surface 110 of the patch 102 can include at least one of the first mating surface 122 and the second mating surface 126. For example, as shown in FIGS. 1 and 2, in some embodiments, at least a portion of the first major surface 110 of the patch 102 can include the second mating surface 126, such that after the flap 106 is wrapped around the medical article 60, the free end 116 of the flap 106 can be secured to the first major surface 110 of the patch 102 by engaging the first mating surface 122 on the first major surface 120 of the flap 106 and the second mating surface 126 on the first major surface 110 of the patch 102. Alternatively, in some embodiments, the patch 102 can include the first mating surface 122, such that the second mating surface 126 on the second major surface 124 of the flap 106 can engage the first mating surface 122 on the patch 102 (e.g., if the flap 106 is further wrapped and tucked under so the second major surface 124 faces the first major surface 110 of the patch 102). Various combinations of these options are also possible.

In embodiments in which the first major surface 110 of the patch 102 includes the first mating surface 122 and/or the second mating surface 126, the mating surface(s) can be provided directly on the first major surface 110 of the patch 102, and/or can be provided by another layer, sheet or material that is coupled to the first major surface 110 of the patch 102.

If securement of the medical article 60 were to be accomplished by engaging the first mating surface 122 on the first major surface 120 of the flap 106 only with the second mating surface 126 on the first major surface 110 of the patch 102, then any displacement or decoupling force exerted on the medical article 60 in the vertical direction V, for example, would initiate a decoupling (i.e., disengagement) of the mechanical fastener 125 predominantly in peel mode. However, in the system 100, the medical article 60 is secured by engaging the complementary first and second mating surfaces 122 and 126 of the mechanical fastener 125 located on the flap 106, as described above. As a result, a similar vertical displacement force exerted on the medical article 60 initiates a decoupling (i.e., disengagement) of the mechanical fastener 125 predominantly in shear mode, i.e., thereby exploiting the relatively greater strength of the mechanical fastener 125 in shear mode, as opposed to in peel mode. As a result, the securement of the medical article 60 can be enhanced, as compared to other securement systems.

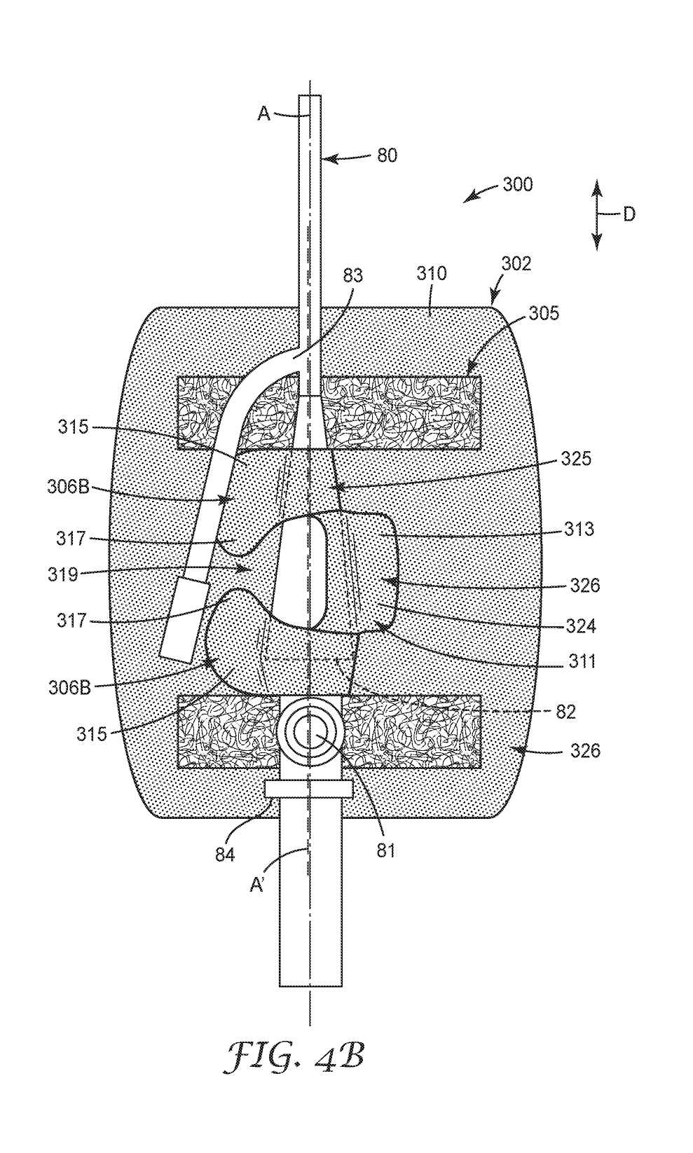

By way of example only, the flap 106 of FIGS. 1 and 2 is shown as being substantially linear and straight. However, in some embodiments, the flap 106 can have a non-linear shape, or include a bulbous end to facilitate being wrapped about medical articles and/or to increase the surface area of the free end 116 of the flap 106 to enhance coupling the flap 106 to the patch 102 (e.g., after being wrapped about a medical article). Such embodiments are described in greater detail below and shown in FIGS. 4A-6B.

In some embodiments, the flap 106 can include a hinge, e.g., a living hinge, about which the free end 116 can pivot to be movable toward and away from a medical article or the patch 102. Such a hinge, for example, can separate the flap 106 from the patch 102 in embodiments in which the flap 106 is integrally formed with the patch 102.

In some embodiments, the flexible flap 106 can provide conformability, ease of handling, ease of application, ease of packaging, universality for many different medical articles, low cost, etc. Furthermore, in some embodiments, the system 100 can be free of any rigid components (e.g., rigid securement devices) that are more rigid than the flap 106. Examples of rigid components, and particularly, rigid securement devices, can include, but are not limited to, one or more of brackets, retainers, clips, posts, clamps, hooks, other typical rigid devices or structures, or a combination thereof.

### Backings

Suitable backings for patches and/or flaps of the present disclosure can include, but are not limited to, one or more of a fabric, a woven fibrous web, a nonwoven fibrous web, a knit, a polymeric film, other familiar dressing materials, or combinations thereof. In some embodiments, the backing materials can include polymeric elastic films (e.g., transparent or non-transparent), and can include, but are not limited to, films formed of elastomeric polyurethanes, co-polyesters, polyethylenes, or combinations thereof. The backing can be a high moisture vapor permeable film, i.e., a backing with a relatively high moisture vapor transmission rate (MVTR). U.S. Pat. No. 3,645,835 describes methods of making such films and methods for testing their permeability. The backing can be constituted of natural or synthetic sources of raw materials.

The backings of patches of the present disclosure advantageously should transmit moisture vapor at a rate equal to or greater than human skin. In some embodiments, the patch backing can be adhesive-coated. In such embodiments, the adhesive-coated backing can transmit moisture vapor at a rate of at least 300 g/m²/24 hrs/37° C./100-10% RH, and in some embodiments, at least 700 g/m²/24 hrs/37° C./100-10% RH. The patch backing is generally conformable to anatomical surfaces. As such, when the patch is applied to an anatomical surface, it conforms to the surface even when the surface is moved.

The backing can be a flexible material. For example, the backing can be a film, paper, woven, knit, foam, nonwoven material, or a combination thereof, or one or more layers of film, paper, woven, knit, foam, nonwoven, or a combination thereof. In some embodiments, it can be desirable that at least a portion of backing is formed of a transparent material to allow for viewing of underlying skin, a medical device, and/or a target site.

By way of example only, in some embodiments, the backing of a patch of the present disclosure can be formed of a film available under the trade designation TEGADERM® from 3M Company, St. Paul, Minn.

Additional exemplary embodiments of medical article securement systems of the present disclosure will now be described with respect to FIGS. 3A-7B. FIGS. 3A-7B illustrate various medical article securement systems of the present disclosure, wherein like numerals represent like elements. The medical article securement systems of FIGS. 3A-7B share many of the same elements, features, and functions as the medical article securement system 100 described above with respect to FIGS. 1-2. Reference is made to the description above accompanying FIGS. 1-2 for a more complete description of the features and elements (and alternatives to such features and elements) of the embodiments illustrated in FIGS. 3A-7B. Any of the features described above with respect to FIGS. 1-2 can be applied to the embodiments of FIGS. 3A-7B, and vice versa.

FIGS. 3A-3B illustrate a medical article securement system 200 according to another embodiment of the present disclosure. The system includes a patch 202 with a skin-contact adhesive 215, and two flaps 206. In some embodiments employing multiple flaps 206, the flaps 206 can form at least a portion of a medical article securement assembly coupled to a first major surface 210 of the patch 202 (i.e., opposite the skin-contact adhesive 215).

The system 200 is substantially the same as the system 100 of FIGS. 1 and 2, except that the system 200 of FIGS. 3A-3B includes two flaps 206—i.e., a first flap 206A and a second flap 206B. FIG. 3A shows the flaps 206 in the first position and FIG. 3B illustrates the flaps 206 in the second position, i.e., at least partially overlapping the medical article 60, and particularly the longitudinal axis A of the medical article 60 (and optionally, a longitudinal axis A′ of the system 300). The same medical article 60 is shown in FIGS. 3A and 3B by way of example only.

As shown in FIGS. 3A and 3B, in some embodiments employing more than one flap 206, the flaps 206 can actually be provided by the same material, or flap, and can be configured to oppose one another. In addition, in some embodiments, the flaps 206 can extend laterally with respect to the longitudinal axis A of the medical article 60 (or of a longitudinal axis A′ of the system 200). By describing the flaps 206 as extending laterally, it should be understood that the flaps 206 need not extend exactly perpendicularly with respect to the longitudinal axis A and/or longitudinal axis A′, but rather that the flaps 206 include a lateral component, such that they extend outwardly with respect to the longitudinal axis A and/or longitudinal axis A′, e.g., even at an acute angle. Said another way, the first flap 206A can be described as extending at least partially in a first lateral direction (e.g., when in its first position, not shown) from the longitudinal axis A and/or longitudinal axis A′, and the second flap 206B can be described as extending in a second lateral direction (e.g., when in its first position, not shown) from the longitudinal axis A and/or longitudinal axis A′, such that the first flap 206A and the second flap 206B are on opposite sides of the longitudinal axis A and/or longitudinal axis A′ and extend at least partially laterally on opposite sides of the longitudinal axis A and/or longitudinal axis A′.

Furthermore, as shown in FIGS. 3A and 3B, in some embodiments, the first flap 206A overlaps the medical article 60 in a first lateral direction L₁ (see FIG. 3B) when moved from its first position to its second position, and the second flap 206B overlaps the medical article 60 in a second lateral direction L₂ (see FIG. 3B) when moved from its first position to its second position. In some embodiments, as shown, the first lateral direction L₁ and the second lateral direction L₂ can be opposite one another. In addition, in some embodiments, the first flap 206A can be on one lateral side of the longitudinal axis A and/or longitudinal axis A′ (and is configured to be wrapped about the medical article 60, e.g., about the circumference of a catheter, in a clockwise direction), and the second flap 206B is on an opposite side of the longitudinal axis longitudinal axis A and/or longitudinal axis A′ from the first flap 206A (and is configured to be wrapped about the medical article 60, e.g., about the circumference of a catheter, in a counter-clockwise direction).

Each flap 206 includes a first major surface 220 configured to face the medical article 60 in the second position, and a second major surface 224 configured to face away from the medical article 60 in the second position. The first major surface 220 of each flap 206 can include a first mating surface 222 of a mechanical fastener 225, and the second major surface 224 of each flap 206 can include a second mating surface 226 of the mechanical fastener 225 configured to engage the first mating surface 222.

In addition, as shown in FIG. 2, the first flap 206A and the second flap 206B can be configured such that the flaps 206 at least partially overlap one another in the second position. As a result, when in their respective second positions, i.e., to secure the medical article 60, the first mating surface 222 of one flap 206 can be positioned to engage the second mating surface 226 of the other flap 206. As shown in FIG. 3B, the flaps 206 can be positioned and arranged such that when the medical article 60 is subjected to a displacement force in the vertical direction V, the shear component of the mechanical fastener 225 can be maximized, exploiting the shear strength of the mechanical fastener 225 as compared to its strength in peel mode.

Furthermore, as shown in FIGS. 3A and 3B, in some embodiments, at least a portion of the first major surface 210 of the patch 202 can include the second mating surface 226 of the mechanical fastener 225, such that the first mating surface 222 on one or both of the flaps 206 can engage the second mating surface 226 on the patch 202 when in the second position to further enhance the securing of the retained medical article 60. In addition, as shown in FIG. 3A, when the flaps 206 are in their respective first positions, the second major surface 224 comprising the second mating surface 226 is positioned to contact the first major surface 210 of the patch 202.

By employing the same mating surface (i.e., the second mating surface 226) on the second major surface 224 of the flaps 206 and the first major surface 210 of the patch 202, the flaps 206 can be inhibited from overly securing to the patch 202 in their first positions (if they come into contact), i.e., to avoid forcing a user to apply significant force (i.e., peel force) to either flap 206 in order to lift it from the patch 202 and secure the medical article 60.

Similar to the system 100 described above, in the system 200, the medical article 60 is secured by engaging the complementary first and second mating surfaces 222 and 226 of the mechanical fastener 225 located on the flaps 206, as described above. As a result, a similar vertical displacement force exerted on the medical article 60 initiates a decoupling (i.e., disengagement of the mechanical fastener 225) predominantly in shear mode, i.e., thereby exploiting the relatively greater strength of the mechanical fastener 225 in shear mode, as opposed to in peel mode. As a result, the securement of the medical article 60 can be enhanced, as compared to other securement systems.

As mentioned above, in some embodiments, the flaps 206 can be formed of the same piece of material, or flap, which can facilitate manufacturing, because the flaps 206 can be formed from a single piece of material having the first mating surface 222 of the mechanical fastener 225 on one side and the second mating surface 226 of the mechanical fastener 225 on the opposite side. Then, the material can be positioned (e.g., on the patch 202) such that when the medical article 60 is secured by the system 200, the material forms two flaps 206 with respect to the medical article 60 that are configured to at least partially overlap one another when in their respective second positions (see FIG. 3B).

In the system 200 of FIGS. 3A and 3B, the first mating surface 222 is shown by way of example only as include piles or loops, and the second mating surface 226 is shown as including hooks or pegs. However, it should be understood that the opposite could be true (e.g., as shown in FIGS. 1 and 2), or any other appropriate mechanical fastener mating surfaces can be employed for either the first mating surface 222 or the second mating surface 226.

Furthermore, while the first flap 206A is shown as being on the left-hand side of FIGS. 3A and 3B, and the second flap 206B is shown as being on the right-hand side of FIGS. 3A and 3B, it should be understood that the designation of “first” and “second” flap is not significant and can be reversed, such that the flap 206A on the left of FIGS. 3A and 3B can be referred to as the second flap, and the flap 206B on the right of FIGS. 3A and 3B can be referred to as the first flap.

FIGS. 4A-4B illustrate a medical article securement system 300 according to another embodiment of the present disclosure. The system includes a patch 302 with a skin-contact adhesive (not shown), and two flaps 306—i.e., a first flap 306A and a second flap 306B.

The system 300 is substantially the same as the system 200 of FIGS. 3A and 3B, except that in the system 300 of FIGS. 4A-4B, at least a portion of the first flap 306A is dimensioned to be received either (i) in a recess of the second flap 306B, and/or (ii) between two second flaps 306B. As such, the medical article securement system of FIGS. 4A-4B shares many of the same elements, features, and functions as the medical article securement system 200 described above with respect to FIGS. 3A-3B. Reference is made to the description above accompanying FIGS. 3A-3B for a more complete description of the features and elements (and alternatives to such features and elements) of the embodiments illustrated in FIGS. 4A-4B. Any of the features described above with respect to FIGS. 3A-3B can be applied to the embodiment of FIGS. 4A-4B, and vice versa.

FIG. 4A shows the flaps 306 in the first position, and FIG. 4B illustrates the flaps 306 in the second position, i.e., at least partially overlapping a medical article 80, and particularly the longitudinal axis A of the medical article 80 (and optionally, a longitudinal axis A′ of the system 300). The medical article 80 of FIGS. 4A and 4B is illustrated by way of example only as being a Foley catheter. The medical article 80 illustrates several examples of irregular features, including a protrusion (e.g., a radial protrusion) 81, a change in diameter 82 or 84, and a multi-lumen joint (e.g., a bifurcation) 83.

As shown in FIGS. 4A and 4B, the flaps 306 can be positioned near at least one irregular feature of the medical article 80, such as one or more of the bifurcation 83, the change in diameter 82, the change in diameter 84, and the protrusion 81, such that longitudinal movement of the medical article 80, i.e., in a longitudinal direction D can be inhibited when the medical article 80 is secured by the system 300.

As shown in FIG. 4A by way of example only, the first flap 306A includes two side portions 307, a central recess 309, and a central tab 311 having a bulbous end 313. By way of further example, the second flap 306B has a complementary shape comprising two side portions 315, each having a bulbous end 317, that are separated by a recess 319, e.g., an open-ended recess 319.

As shown, a first major surface 320 of the flaps 306 can include a first mating surface 322 of a mechanical fastener 325 (see FIG. 4A, shown by way of example only as piles or loops), and a second major surface 324 of the flaps 306 can include a second mating surface 326 of the mechanical fastener 325 (see FIG. 4B, shown by way of example only as hooks or pegs).

In addition, in some embodiments, as shown in FIGS. 4A and 4B, the patch 302 can include the second mating surface 326 of the mechanical fastener 325 on a first major surface 310 thereof. Furthermore, as shown by way of example only, in some embodiments, the system 300 can further include one or more coupling or anchoring layers or portions 305 that optionally can be integrally formed with the flaps 306 and which can provide coupling means for securing and anchoring the flaps 306 to the patch 302. Such an embodiment can be useful, e.g., when the flaps 306 are not integrally formed with the patch 302. In such embodiments, a specific patch 302 for a particular application (e.g., having the desired material properties, such as moisture vapor transfer rate, transparency, etc.) can be employed, and the flaps 306 can be coupled to the patch 302.

The coupling layer 305 can be coupled to the patch 302 by any of the above-described coupling means, and is shown in FIGS. 4A and 4B by way of example only as being integrally formed with the flaps 306 for simplicity. That is, in the embodiment illustrated in FIGS. 4A and 4B, the flaps 306 and the coupling layer 305 are formed of the same material, having the first mating surface 322 on the first major surface 320 and the second mating surface 326 on the opposite, second major surface 324.

In addition, by way of example only, the coupling layer 305 is shown as including two lateral strips or bars that are spaced longitudinally apart—i.e., distally and proximally with respect to the flaps 306. However, other configurations and arrangements are possible. In addition, in some embodiments, the flaps 306 can be directly coupled to the patch 302 or can be integrally formed with the patch 302, and the coupling layer 305 is shown merely for illustrative purposes.

Still, in other embodiments, the portion of the system 300 illustrated as the coupling layer 305 can instead form the “patch” of the system 300, such that the illustrated patch 302 is not necessary. Rather, in such embodiments, the coupling layer 305 would function as the patch (i.e., having the desired qualities and skin-contact adhesive for being adhered to skin), and the flaps 306 can be coupled thereto or integrally formed therewith.

In use, the medical article 80 can be positioned on the system 300 such that the longitudinal axis A of the medical article 80 aligns with the longitudinal axis A′ of the system 300, as shown in FIG. 4A. Then, as shown in FIG. 4B, the first flap 306A can be folded over at least a portion of (e.g., at least a portion of a circumference of) the medical article 80, such that the tab 311 of the first flap 306A is received in the recess 319 of the second flap 306B. The first mating surface 322 on the first major surface 320 of the tab 311 (e.g., on the bulbous end 313 thereof) of the first flap 306A can be further secured to the second mating surface 326 on the patch 302, as shown in FIG. 4B.

Then, as further shown in FIG. 4B, the second flap 306B can be folded over at least a portion of the medical article 80, generally opposite the first flap 306A. As a result of the complementary shape and size of the second flap 306B, the two side portions 315 of the second flap 306B overlap the two side portions 307 of the first flap 306A (e.g., over an upper surface of the medical article 80), such that the shear strength of the mechanical fastener 325 is exploited. In addition, in some embodiments, at least a portion of the tab 311 of the first flap 306A can also overlap a central portion 331 of the second flap 306B when the flaps 306 are in their respective second positions. The side portions 315, and particularly, the bulbous ends 317 thereof, of the second flap 306B can optionally be further secured to the second mating surface 326 on the patch 302 (if employed).

While the flaps 306 are described as including two side portions 307 and 315, it should be understood that one or more than two such overlapping portions can instead be employed, and that the shape illustrated in FIGS. 4A and 4B is shown by way of example only, as long as the flaps 306 are complementary in shape and size, such that the first mating surface 322 of one flap 306 overlaps and engages with the second mating surface 326 of another flap 306 (i.e., in shear mode) so as to form a reliable securement system.

Similar to the systems 100 and 200 described above, in the system 300, the medical article 60 is secured by engaging the complementary first and second mating surfaces 322 and 326 of the mechanical fastener 325 located on the flaps 306, as described above. As a result, a similar vertical displacement force exerted on the medical article 60 initiates a decoupling (i.e., disengagement of the mechanical fastener 325) predominantly in shear mode, i.e., thereby exploiting the relatively greater strength of the mechanical fastener 325 in shear mode, as opposed to in peel mode. As a result, the securement of the medical article 60 can be enhanced, as compared to other securement systems.

While the second flap 306B is described above as having two side portions 315 that overlap and engage with portions of the first flap 306A, it should be understood that the flaps 306 of the system 300 can instead be described as including a plurality of second flaps 306B (i.e., two second flaps 306B). In such embodiments, the two second flaps 306B can be longitudinally spaced apart to define the recess 319, and the first flap 306A can be described as having at least a portion that is dimensioned to be received between two second flaps 306B (i.e., between two adjacent second flaps 306B).

In addition, the bulbous ends 313 and 317 of the flaps 306 are shown by way of example only to illustrate that the flaps 306 need not be perfectly straight or linear, and that, in some embodiments, it can be advantageous for the flaps 306 to have a non-linear, arcuate, or otherwise irregular shape to facilitate being wrapped about the medical article 80 and being further secured to the first major surface 310 of the patch 302.

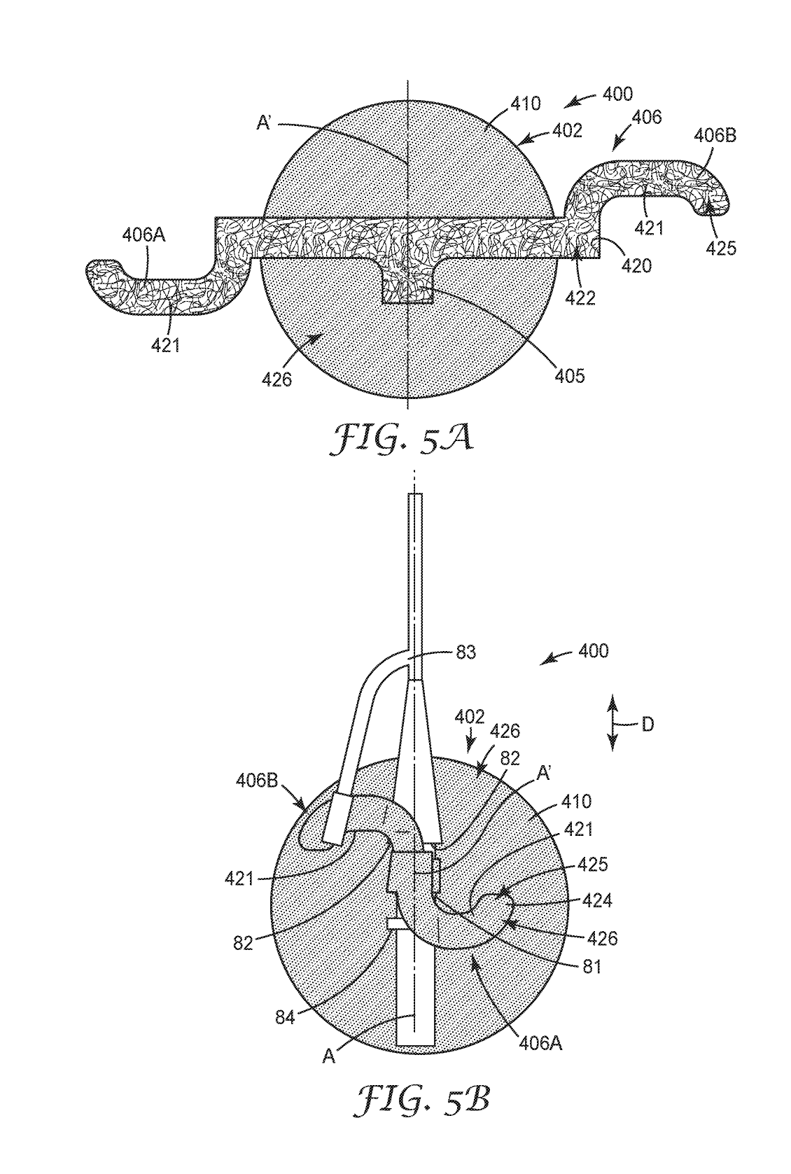

FIGS. 5A-5B illustrate a medical article securement system 400 according to another embodiment of the present disclosure. The system includes a patch 402 with a skin-contact adhesive (not shown), and two flaps 406—i.e., a first flap 406A and a second flap 406B.

The system 400 is substantially the same as the system 200 of FIGS. 3A-3B and the system 300 of FIGS. 4A-4B, except that in the system 400 of FIGS. 5A-5B, each of the first flap 406A and the second flap 406B includes a non-linear or irregular shape, and particularly includes an arcuate end portion 421, which can facilitate being wrapped about at least a portion of the medical article 80 and/or being coupled to a first major surface 410 of the patch 402. As such, the medical article securement system of FIGS. 5A-5B shares many of the same elements, features, and functions as the medical article securement systems 200 and 300 described above with respect to FIGS. 3A-3B and 4A-4B, respectively. Reference is made to the description above accompanying FIGS. 3A-3B and 4A-4B for a more complete description of the features and elements (and alternatives to such features and elements) of the embodiment illustrated in FIGS. 5A-5B. Any of the features described above with respect to FIGS. 3A-3B and 4A-4B can be applied to the embodiment of FIGS. 5A-5B, and vice versa.

FIG. 5A shows the flaps 406 in the first position, and FIG. 5B illustrates the flaps 406 in the second position, i.e., at least partially overlapping a medical article 80, and particularly the longitudinal axis A of the medical article 80 (and optionally, a longitudinal axis A′ of the system 300). The medical article 80 is shown in FIG. 5B by way of example only as being a Foley catheter—i.e., the same medical article 80 as shown in FIGS. 4A-4B.

As shown in FIG. 5B, the flaps 406 can be positioned near at least one of the irregular features of the medical article 80, i.e., near at least one of the bifurcation 83, the change in diameter 82, the change in diameter 84, and the protrusion 81, such that longitudinal movement of the medical article 80, i.e., in a longitudinal direction D can be inhibited when the medical article 80 is secured by the system 400. The flaps 406 are shown as being positioned adjacent the change in diameter 82 of the medical article 80 by way of example only.

As shown, a first major surface 420 of the flaps 406 can include a first mating surface 422 of a mechanical fastener 425 (see FIG. 5A, shown by way of example only as piles or loops), and a second major surface 424 of the flaps 406 can include a second mating surface 426 of the mechanical fastener 425 (see FIG. 5B, shown by way of example only as hooks or pegs).

As shown in FIGS. 5A and 5B, the patch 402 can include the second mating surface 426 of the mechanical fastener 425 on a first major surface 410 thereof. Furthermore, in some embodiments, the system 400 can further include one or more coupling or anchoring layers or portions 405 (see FIG. 5A) that can be integrally formed with the flaps 406 and which can provide coupling means for securing and anchoring the flaps 406 to the patch 402. The small, centrally located and longitudinally extending coupling layer 405 is shown by way of example only. As described above with respect to the system 300 of FIGS. 4A and 4B, in some embodiments, the system 400 need not include the coupling layer 405. Alternatively, in some embodiments, the coupling layer 405 shown in FIG. 5A can function as the “patch” of the system 400, and the system 400 need not include the illustrated patch 402.

In use, the medical article 80 can be positioned on the system 400 such that the longitudinal axis A of the medical article 80 aligns with the longitudinal axis A′ of the system 400, as shown in FIG. 5B. Then, the first flap 406A or the second flap 406B can be folded over at least a portion of (e.g., at least a portion of a circumference of) the medical article 80. By way of example only, FIG. 5B shows the second flap 406B as being positioned over the medical article 80 first, followed by the first flap 406A.

Then, as further shown in FIG. 5B, the first flap 406A can be folded over at least a portion of the medical article 80, generally opposite the second flap 406B. As a result of the complementary shape, size, and relative positioning of the flaps 406, at least a portion of the first flap 406A overlaps at least a portion of the second flap 406B (or vice versa), such that the shear strength of the mechanical fastener 425 is exploited. One or both of the arcuate portions 421 of the flaps 406 can optionally be further secured to the second mating surface 426 on the patch 402 (if employed), as shown in FIG. 5B.

Similar to other systems described above, when in the second position, the flaps 406 are coupled to one another in a way that exploits the shear strength of the mechanical fastener 425 (i.e., decoupling in shear mode). As a result, the securement of the medical article 80 can be enhanced.

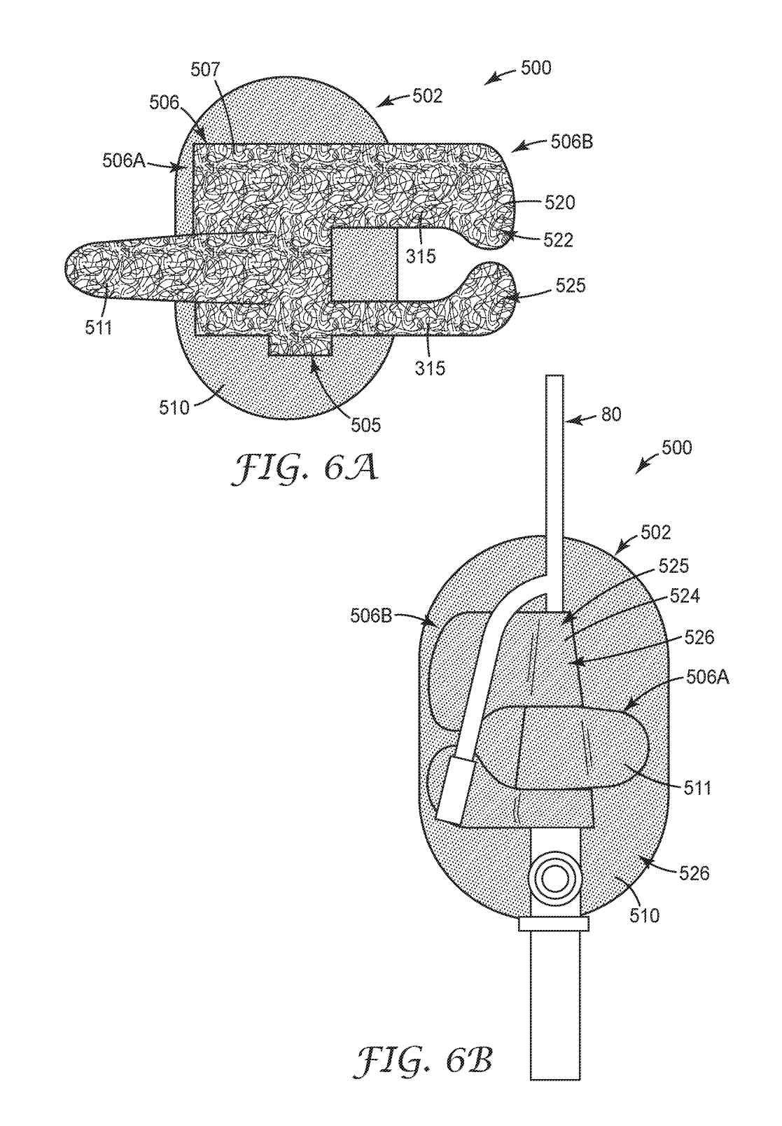

While the flaps 406 are each illustrated as including an arcuate portion 421, it should be understood that the flaps 406 of the system 300 can include any necessary shape to enhance coupling a particular medical article and/or to enhance coupling the free ends of the flaps 406 to the patch 402. That is, the flaps 406 are shown by way of example only to illustrate that the flaps 406 need not be perfectly straight or linear, and that, in some embodiments, it can be advantageous for the flaps 406 to have a non-linear, or otherwise irregular shape to facilitate being wrapped about the medical article 80 and being further secured to the first major surface 410 of the patch 402. Such irregular flaps 406 may also help distribute stress across the flap 406 and/or the patch 402. FIGS. 6A-6B illustrate a medical article securement system 500 according to another embodiment of the present disclosure. The system includes a patch 502 with a skin-contact adhesive (not shown), and two flaps 506—i.e., a first flap 506A and a second flap 506B.

The system 500 is substantially the same as the system 300 of FIGS. 4A-4B, except that the system 500 of FIGS. 6A-6B illustrates a different shape and configuration for the flaps 506 and a coupling layer 505 (if employed). Particularly, the first flap 506A does not include a recess or a bulbous end, but does include a base portion 507 and a tab (or extension) 511 that is longer than the base portion 307 to be wrapped further about the medical article 80 and optionally into contact with the patch 502 on the opposite side of the medical article 80 (as shown in FIG. 6B). In some embodiments, as shown in FIGS. 6A and 6B, the tab 511 can be located within a longitudinal length of the base portion 507, such that the base portion 507 is present on a proximal and a distal side of the tab 511. In such embodiments, the base portion 507 can be referred to as two side portions 507.

As a result, the medical article securement system of FIGS. 5A-5B shares many of the same elements, features, and functions as at least the medical article securement system 300 described above with respect to FIGS. 4A-4B. Reference is made to the description above accompanying FIGS. 4A-4B for a more complete description of the features and elements (and alternatives to such features and elements) of the embodiment illustrated in FIGS. 6A-6B. Any of the features described above with respect to FIGS. 4A-4B can be applied to the embodiment of FIGS. 6A-6B, and vice versa.

FIG. 6A shows the flaps 506 in the first position, and FIG. 6B illustrates the flaps 506 in the second position. The medical article 80 is shown in FIG. 5B by way of example only as being a Foley catheter—i.e., the same medical article 80 as shown in FIGS. 4A-4B. Similar to the system 300 of FIGS. 4A-4B, the second flap 506B can be described as having two side portions 515 and a recess 319 formed therebetween, or the system 500 can be described as having two second flaps 506B, longitudinally separated by a recess 519.

As shown in FIG. 6B, the flaps 506 can be positioned near at least one of the irregular features of the medical article 80. As further shown, a first major surface 520 of the flaps 506 can include a first mating surface 522 of a mechanical fastener 525 (see FIG. 6A, shown by way of example only as piles or loops), and a second major surface 524 of the flaps 506 can include a second mating surface 526 of the mechanical fastener 525 (see FIG.6B, shown by way of example only as hooks or pegs).

The small, centrally located and longitudinally extending coupling layer 505 is shown by way of example only. As described above with respect to the system 300 of FIGS. 4A and 4B, in some embodiments, the system 500 need not include the coupling layer 505. Alternatively, in some embodiments, the coupling layer 505 shown in FIG. 6A can function as the “patch” of the system 500, and the system 500 need not include the illustrated patch 502.

In use, the medical article 80 can be positioned on the system 500 such that the longitudinal axis A of the medical article 80 aligns with the longitudinal axis A′ of the system 500, as shown in FIG. 6B. Then, the first flap 506A can be folded over at least a portion of (e.g., at least a portion of a circumference of) the medical article 80. Then, as further shown in FIG. 5B, the second flap 506B can be folded over at least a portion of the medical article 80, generally opposite the first flap 506A. As a result of the complementary shape, size, and relative positioning of the flaps 506 (e.g., the base portion 507 of the first flap 506A having a shorter lateral dimension than the complementary portions of the second flap 506B), at least a portion of the second flap 506B overlaps at least a portion of the first flap 506A (and/or vice versa), such that the shear strength of the mechanical fastener 525 is exploited. At least a portion of one or both of the flaps 506 can optionally be further secured to a second mating surface 526 on the patch 502 (if employed), as shown in FIG. 6B.

Similar to other systems described above, when in the second position, the flaps 506 are coupled to one another in a way that exploits the shear strength of the mechanical fastener 525 (i.e., decoupling in shear mode). As a result, the securement of the medical article 80 can be enhanced.

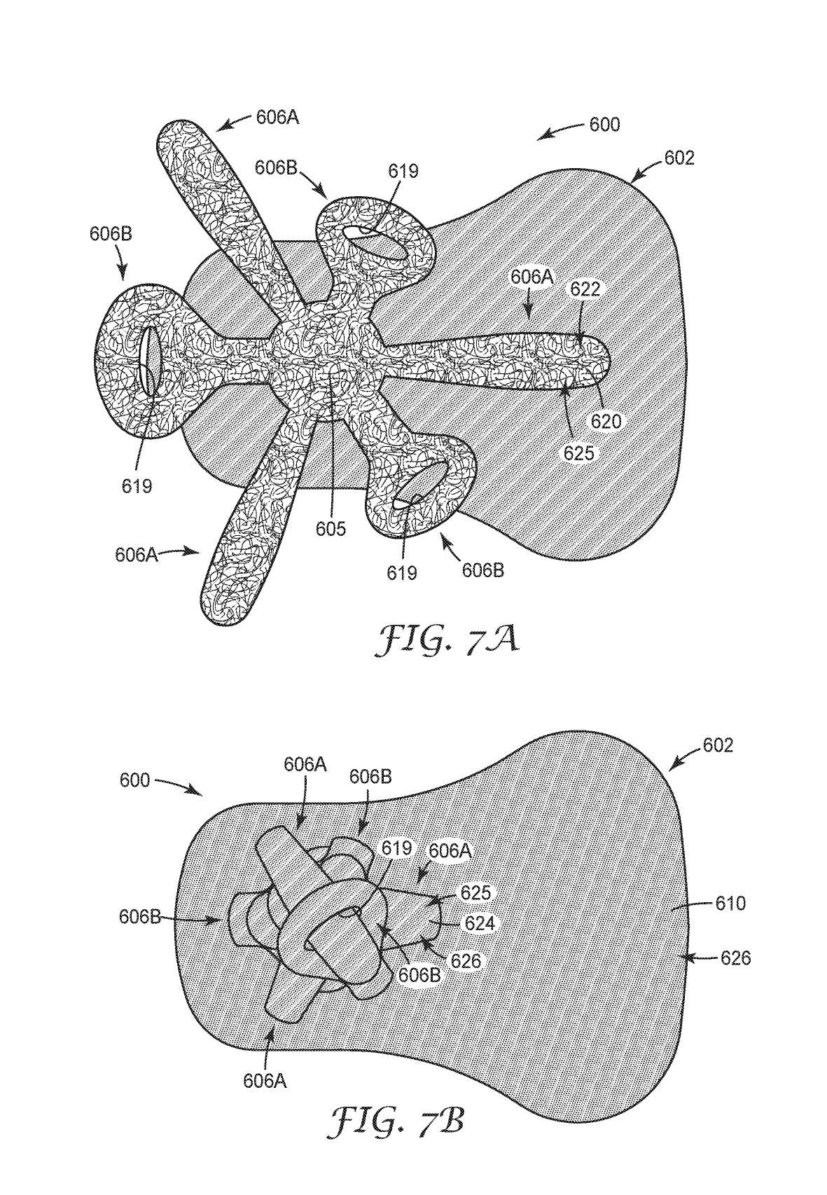

FIGS. 7A-7B illustrate a medical article securement system 600 according to another embodiment of the present disclosure. The system includes a patch 602 with a skin-contact adhesive (not shown), and six flaps 606—three sets of a first flap 606A and a second flap 606B located across a hub 605 from one another. The hub 605 can function as a coupling layer, similar to other embodiments described above, which can in turn be coupled to the patch 602. However it should also be understood that one or more of the flaps 606 can instead be integrally formed with the patch 602, or the hub 605 can function as the “patch” of the system 600.

FIG. 7A shows the flaps 606 in the first position, and FIG. 6B illustrates the flaps 606 in the second position. No medical article is shown in FIGS. 7A-7B for simplicity, but it should be understood that any medical article of interest can be secured with the system 600, and the system 600 can be particularly configured for a particular medical article. When in the first position, as shown in FIG. 7A, each of the flaps 606 extends radially outwardly from the hub 605 (which is shown as being circular by way of example only). By way of example, the system 600 includes three sets of first and second flaps 606A and 606B, and each of the flaps 606 are equally spaced apart about the hub 605, such that the flaps 606 are each separated by 60 degrees from an adjacent flap 606. This number and configuration of flaps 606 are shown by way of example only, and it should be understood that as few as one set of opposing first and second flaps 606A and 606B and as many sets as possible can be employed.

By way of example, each first flap 606A widens as it extends radially outwardly from the hub 605, and at least a portion of each first flap 606A is dimensioned to be received in an aperture or recess 619 of an opposing second flap 606B. Accordingly, each second flap 606B has an aperture or recess 619 dimensioned to receive at least a portion of the respective, opposing first flap 606A.

As described above with other systems of the present disclosure, one or more sets of flaps 606 can be positioned near at least one irregular feature of a medical article to aid in securing the medical article. As further shown, a first major surface 620 of the flaps 606 can include a first mating surface 622 of a mechanical fastener 625 (see FIG. 7A, shown by way of example only as piles or loops), and a second major surface 624 of the flaps 606 can include a second mating surface 626 of the mechanical fastener 625 (see FIG.7B, shown by way of example only as hooks or pegs).

By way of example only, the flaps 606 and the coupling layer 605 are all shown as being formed of the same material, such that the flaps 606 all have the same first mating surface 622 on the first major surface 620 thereof, and the flaps 606 all have the same second mating surface 626 on the second major surface 624 thereof. This can enhance securing of a medical article, because when the first major surface 620 of any flap 606 is folded over into its second position into contact with a second major surface 624 of itself or another flap 606, the first mating surface 622 and the second mating surface 626 can engage. However, this need not be the case, and in some embodiments, at least one of the sets of first and second flaps 606A and 606B can include a different mechanical fastener 625 that may or may not engage with one or more other flap sets.

In use, a medical article can be positioned on the system 600 (e.g., on the patch 602 and/or on the coupling layer 605). Then, the first flap 606A of a first flap set can be folded over at least a portion of (e.g., at least a portion of a circumference of) the medical article and threaded at least partially through the aperture 619 of the opposing second flap 606B. In doing so, the second flap 606B can also be folded over at least a portion of the medical article, generally opposite its respective first flap 606A. As a result of the complementary shape, size, and relative positioning of the flaps 606, at least a portion of the first flap 606A overlaps at least a portion of the opposing second flap 606B (e.g., after passing through the aperture 619), and at least a portion of the second flap 606B overlaps at least a portion of the first flap 606A (e.g., at least a portion of the second flap 606B comprising the aperture 619 can be positioned to overlap the second major surface 624 of the opposing first flap 606A), such that the shear strength of the mechanical fastener 625 is exploited. The remaining sets of first and second flaps 606A and 606B can then be secured in a similar manner. The sequential securing of sets of flaps 606 can allow one or more flaps 606 from one set to overlap and engage with one or more flaps 606 of one or more other sets.

At least a portion of any of the flaps 606 (e.g., at least a portion of one or more first flaps 606A after passing through the aperture 619 of an opposing second flap 606B) can optionally be further secured to a second mating surface 626 on the patch 602 (if employed), as shown in FIG. 7B.

Similar to other systems described above, when in the second position, the flaps 606 are coupled to one another in a way that exploits the shear strength of the mechanical fastener 625 (i.e., decoupling in shear mode). As a result, the securement of a medical article can be enhanced.

Each embodiment shown in the figures is illustrated as a separate embodiment for clarity in illustrating a variety of features of the medical article securement systems of the present disclosure. However, it should be understood that any combination of elements and features of any of the embodiments illustrated in the figures and described herein can be employed in the medical article securement systems of the present disclosure.

The following embodiments are intended to be illustrative of the present disclosure and not limiting.

### Embodiments

Embodiment 1 is a medical article securement system for securing a medical article, the system comprising:
a patch comprising
   a first major surface, and
   a second major surface, opposite the first major surface, comprising a skin-contact adhesive; and
at least one flap comprising
   a fixed end coupled to the patch, and
   a free end that is movable with respect to the patch and configured to secure at least a portion of the medical article to the patch, at least the free end comprising:
      a first major surface configured to face the medical article, at least a portion of the first major surface comprising a first mating surface of a mechanical fastener, and
      a second major surface, opposite the first major surface, configured to face away from the medical article, at least a portion of the second major surface comprising a second mating surface of the mechanical fastener configured to engage the first mating surface of the same flap, a different flap, or both.

Embodiment 2 is the system of embodiment 1, wherein the first mating surface and the second mating surface are positioned to engage one another adjacent at least an upper surface of the medical article, when the at least one flap is coupled to the medical article. Embodiment 3 is the system of embodiment 1 or 2, wherein the free end of the at least one flap is movable with respect to the patch and the fixed end of the flap between a first position in which the at least a portion of the free end is not positioned in an overlapping relationship with the medical article and a second position in which at least a portion of the free end is positioned in an overlapping relationship with the medical article.

Embodiment 4 is the system of any of embodiments 1-3, wherein the at least one flap includes a first flap positioned to at least partially overlap a second flap, such that the first mating surface of the first flap and the second mating surface of the second flap are positioned to engage one another.

Embodiment 5 is the system of embodiment 4, wherein the first flap and the second flap each extend laterally with respect to a longitudinal axis of the system.

Embodiment 6 is the system of embodiment 4 or 5, wherein the first flap and the second flap oppose one another.

Embodiment 7 is the system of any of embodiments 4-6, wherein the first flap extends at least partially in a first lateral direction from a longitudinal axis of the system, and the second flap extends at least partially in a second lateral direction from the longitudinal axis that is different from the first lateral direction, such that the first flap and the second flap are on opposite sides of the longitudinal axis.

Embodiment 8 is the system of any of embodiments 4-7, wherein at least a portion of the first flap is dimensioned to be received in a recess of the second flap.

Embodiment 9 is the system of any of embodiments 4-8, wherein the second flap is one of a plurality of second flaps, and wherein at least a portion of the first flap is dimensioned to be received between two second flaps.

Embodiment 10 is the system of embodiment 9, wherein the second flaps are separated a longitudinal distance from one another.

Embodiment 11 is the system of any of embodiments 4-10, wherein the first flap is on one side of a longitudinal axis of the system, and the second flap is on an opposite side of the longitudinal axis from the first flap.

Embodiment 12 is the system of any of embodiments 4-11, wherein the second flap includes a recess dimensioned to receive at least a portion of the first flap.

Embodiment 13 is the system of any of embodiments 1-12, wherein at least one flap extends laterally from its fixed end to its free end with respect to the longitudinal axis.

Embodiment 14 is the system of any of embodiments 1-13, wherein the free end of at least one flap is movable with respect to the patch between a first position in which the free end does not cross a longitudinal axis of the system and a second position in which the free end crosses the longitudinal axis of the system.

Embodiment 15 is the system of any of embodiments 1-14, wherein the medical article includes an irregular feature, and wherein at least one flap is configured to be coupled to a portion of the medical article comprising or adjacent the irregular feature.

Embodiment 16 is the system of any of embodiments 1-15, wherein at least one flap has a non-linear shape.

Embodiment 17 is the system of any of embodiments 1-16, wherein at least one flap is integrally formed with the patch.

Embodiment 18 is the system of any of embodiments 1-17, wherein the medical article includes a medical tubing, and wherein at least one flap is configured to be wrapped about at least a portion of the circumference of the medical tubing.

Embodiment 19 is the system of any of embodiments 1-18, wherein the medical article includes a medical tubing and wherein at least one flap is configured to be wrapped about the circumference of the medical tubing at least one time.

Embodiment 20 is the system of any of embodiments 1-19, wherein the system is free of a rigid component that is more rigid than the at least one flap.

Embodiment 21 is the system of any of embodiments 1-20, wherein the first major surface of the patch includes at least one of the first mating surface and the second mating surface.

Embodiment 22 is the system of any of embodiments 1-21, wherein at least one flap is movable with respect to the patch between a first position in which the free end does not overlap a longitudinal axis of the system and a second position in which the free end overlaps the longitudinal axis of the system.

Embodiment 23 is a medical article securement system for securing a medical article, the system comprising:
a longitudinal axis configured to be substantially aligned with a longitudinal axis of the medical article;
a patch comprising
   a first major surface configured to receive the medical article, and
   a second major surface, opposite the first major surface, comprising a skin-contact adhesive; and
at least one flap comprising
   a fixed end coupled to the patch, and
   a free end that is movable with respect to the patch and configured to be wrapped around at least a portion of the medical article to couple at least a portion of the medical article to the patch, the free end comprising:
      a first major surface configured to face the medical article, at least a portion of the first major surface comprising a first mating surface of a mechanical fastener, and
      a second major surface, opposite the first major surface, configured to face away from the medical article, at least a portion of the second major surface comprising a second mating surface of the mechanical fastener configured to engage the first mating surface of the same flap, a different flap, or both, when coupled to the medical article.

Embodiment 24 is a medical article securement system for securing a medical article, the system comprising:
a longitudinal axis configured to be substantially aligned with a longitudinal axis of the medical article;
a patch comprising
   a first major surface, and
   a second major surface, opposite the first major surface, comprising a skin-contact adhesive; and
a medical article securement assembly coupled to the first major surface of the patch, the medical article securement assembly comprising:
   a first flap, and
   a second flap, wherein each of the first flap and the second flap include:
      a fixed end coupled to the patch, and
      a free end that is movable with respect to the patch between a first position in which the free end does not overlap the longitudinal axis of the system and a second position in which the free end overlaps the longitudinal axis of the system,
      a first major surface configured to face the medical article when the free end is in the second position, at least a portion of the first major surface comprising a first mating surface of a mechanical fastener and
      a second major surface, opposite the first major surface, at least a portion of the second major surface comprising a second mating surface of the mechanical fastener, the second mating surface configured to engage the first mating surface of at least one of the first flap and the second flap;
   wherein the first flap extends at least partially in a first lateral direction from the longitudinal axis, and the second flap extends in a second lateral direction from the longitudinal axis, such that the first flap and the second flap are on opposite sides of the longitudinal axis at least when the first flap and the second flap are each in the first position.

Embodiment 25 is the system of embodiment 24, wherein the medical article securement assembly includes at least one of:
a plurality of first flaps, and
a plurality of second flaps.

Embodiment 26 is the system of embodiment 24 or 25, wherein the first flap and the second flap are positioned in an overlapping relationship with the medical article when the free ends of the first flap and the second flap are each in the second position.

It is to be understood that the invention is not limited in its application to the details of construction and the arrangement of components set forth in the above description or illustrated in the accompanying drawings. The invention is capable of other embodiments and of being practiced or of being carried out in various ways. Also, it is to be understood that the phraseology and terminology used herein is for the purpose of description and should not be regarded as limiting. It is to be further understood that other embodiments may be utilized, and structural or logical changes may be made without departing from the scope of the present disclosure.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure.

All references and publications cited herein are expressly incorporated herein by reference in their entirety into this disclosure.

Various features and aspects of the present disclosure are set forth in the following claims.

1. A medical article securement system for securing a medical article, the system comprising:
a patch comprising
a first major surface, and
a second major surface, opposite the first major surface, comprising a skin-contact adhesive; and
at least one flap comprising
a fixed end coupled to the patch, and
a free end that is movable with respect to the patch and configured to secure at least a portion of the medical article to the patch, at least the free end comprising:
a first major surface configured to face the medical article, at least a portion of the first major surface comprising a first mating surface of a mechanical fastener, and
a second major surface, opposite the first major surface, configured to face away from the medical article, at least a portion of the second major surface comprising a second mating surface of the mechanical fastener configured to engage the first mating surface of the same flap, a different flap, or both.

2. The system of claim 1, wherein the first mating surface and the second mating surface are positioned to engage one another adjacent at least an upper surface of the medical article, when the at least one flap is coupled to the medical article.

3. The system of claim 1, wherein the free end of the at least one flap is movable with respect to the patch and the fixed end of the flap between a first position in which the at least a portion of the free end is not positioned in an overlapping relationship with the medical article and a second position in which at least a portion of the free end is positioned in an overlapping relationship with the medical article.

4. The system of claim 1, wherein the at least one flap includes a first flap positioned to at least partially overlap a second flap, such that the first mating surface of the first flap and the second mating surface of the second flap are positioned to engage one another.

5. The system of claim 4, wherein the first flap and the second flap each extend laterally with respect to a longitudinal axis of the system.

6. The system of claim 4, wherein the first flap and the second flap oppose one another.

7. The system of claim 1, wherein the first flap extends at least partially in a first lateral direction from a longitudinal axis of the system, and the second flap extends at least partially in a second lateral direction from the longitudinal axis that is different from the first lateral direction, such that the first flap and the second flap are on opposite sides of the longitudinal axis.

8. The system of claim 1, wherein at least a portion of the first flap is dimensioned to be received in a recess of the second flap.

9. The system of claim 1, wherein the second flap is one of a plurality of second flaps, and wherein at least a portion of the first flap is dimensioned to be received between two second flaps.

10. The system of claim 1, wherein at least one flap extends laterally from its fixed end to its free end with respect to the longitudinal axis.

11. The system of claim 1, wherein the free end of at least one flap is movable with respect to the patch between a first position in which the free end does not cross a longitudinal axis of the system and a second position in which the free end crosses the longitudinal axis of the system.

12. The system of claim 1, wherein the medical article includes an irregular feature, and wherein at least one flap is configured to be coupled to, or adjacent, the irregular feature.

13. The system of claim 1, wherein at least one flap has a non-linear shape.

14. The system of claim 1, wherein at least one flap is integrally formed with the patch.

15. The system of claim 1, wherein the medical article includes a medical tubing, and wherein at least one flap is configured to be wrapped about at least a portion of the circumference of the medical tubing.

16. The system of claim 1, wherein the medical article includes a medical tubing and wherein at least one flap is configured to be wrapped about the circumference of the medical tubing at least one time.

17. The system of claim 1, wherein the system is free of a rigid component that is more rigid than the at least one flap.

18. The system of claim 1, wherein the first major surface of the patch includes at least one of the first mating surface and the second mating surface.

19. The system of claim 2, wherein the free end of the at least one flap is movable with respect to the patch and the fixed end of the flap between a first position in which the at least a portion of the free end is not positioned in an overlapping relationship with the medical article and a second position in which at least a portion of the free end is positioned in an overlapping relationship with the medical article.

20. The system of claim 2, wherein the at least one flap includes a first flap positioned to at least partially overlap a second flap, such that the first mating surface of the first flap and the second mating surface of the second flap are positioned to engage one another.
